(19) United States
(12) Patent Application Publication
(10) Pub. No.: US 2011/0165276 A1
(43) Pub. Date: Jul. 7, 2011

(54) COMPOSITION FOR BURN TREATMENT

(76) Inventor: Rufus Coley, Staley, NC (US)

(21) Appl. No.: 12/858,792

(22) Filed: Aug. 18, 2010

Related U.S. Application Data
(60) Provisional application No. 61/235,170, filed on Aug. 19, 2009.

(51) Int. Cl. A61K 36/886; A61P 17/02

(52) U.S. Cl. 424/744

(57) ABSTRACT

The present invention relates a new composition based on a mixture of a solvent, aloe vera, and sulfur. More particularly the present invention describes a solvent that contains a certain arrangement of minerals which when combined with solid sulfur and aloe vera provide a topical solution that can alleviate pain and rapidly progress healing time of burns of the skin.

### FIELD OF INVENTION

This invention relates generally to skin products and particularly to emulsion-based skin products that can be applied to the skin to provide continuing antibacterial, antiviral, antiseptic, and skin protecting or healing properties.

### BACKGROUND OF INVENTION

The present invention relates generally to preparations for treating the skin and, more particularly, to a preparation for treatment of the skin that contains a high concentration of sulfur.

Sulfur is known to have properties that aid in the treatment of dermatitic conditions. It is employed in various forms for its germicidal and fungicidal actions.

Aloe vera is known to aid in the healing of burns and abrasions to the skin when applied topically. It is also know to reduce local inflammation of the skin.

Topical antimicrobials are currently prescribed by healthcare providers to prevent and treat a variety of serious skin infections such as impetigo, infected diabetic ulcers, venous stasis ulcers, infected surgical wounds, burns, acne, psoriasis and other topical infections, increasingly, topical antimicrobials that contain antibiotics are not effective against microbes which have developed drug resistance

A skin-care product could be formulated to topically deliver medicinally active agents to burns and wounds. Optimum wound or burn care products should reduce inflammation by delivering anti-inflammatory agents or biological precursors of anti-inflammatory agents.

What is needed is a composition useful in treating burns on the skin to reduce or prevent scarring, as well as alleviate pain caused by the burns. Furthermore, a composition is needed for treatment of burned areas on the skin to eliminate bacteria and microorganisms to prevent infection and disease at the site of the exposed skin due to the burns.

### SUMMARY OF INVENTION

The present invention relates to a composition of a water based solvent, aloe vera, and solidified sulfur. This combination has the ability to alleviate pain and increase healing of a burned area on the skin. In combination the aloe vera and sulfur have the ability to reduce inflammation at the site of the burn, as well as increase the natural healing process of the skin to shorten the healing process. The aloe vera increases the growth and effectiveness of the skin fibroblasts, which allow the skin to grow back and heal the site. The increased activity of the fibroblasts around the burn site causes less or in some cases no scarring of the tissue and skin, leaving good healthy skin.

One aspect of the present invention contemplates a composition, comprising of a solvent, in association with sulfur, and aloe vera, combined to treat burns incurred to the skin by chemicals, the sun or another situation that could cause burns.

Another aspect of the present invention contemplates a composition, comprising solvent, in association with sulfur, and aloe vera, wherein the combination of the components are to be applied topically.

Another aspect of the present invention contemplates a composition, comprising solvent, in association with sulfur, and aloe vera, wherein the solvent is water based.

Another aspect of the present invention contemplates a composition, comprising solvent, in association with sulfur, and aloe vera, wherein a water based solvent comprises of a) 400.4 parts per million of Calcium, b) 310 parts per million of Sulfur, c) 260.4 parts per million of Copper, d) 16.5 parts per million of Gold, e) 13.6 parts per million of Silver, f) 12.2 parts per million of Iron.

Another aspect of the present invention contemplates a composition, comprising solvent, in association with sulfur, and aloe vera, wherein the sulfur is in the solidified state.

Another aspect of the present invention contemplates a composition, comprising solvent, in association with sulfur, and aloe vera, wherein the aloe vera is an extract from the aloe vera plant in the gel or liquid form so as to be mixed with the water based solvent.

### DETAILED DESCRIPTION

### Definitions and Use of Terms

The present invention may be understood more readily by reference to the following detailed description of preferred embodiments of the invention.

Before the present compounds, compositions and methods are disclosed and described, it is to be understood that this invention is not limited to specific synthetic methods, or to specific formulations or administration regimens, as such may, of course, vary. It is also to be understood that the terminology used herein is for the purpose of describing particular embodiments only and is not intended to be limiting.

It must be noted that, as used in the specification and the appended claims, the singular forms “a”, “an”, and “the” include plural referents unless the context clearly dictates otherwise.

Ranges are often expressed herein as from about one particular value, and/or to about another particular value. When such a range is expressed, it is to be understood that a more preferred range is typically from the one particular value and/or to the other particular value. Similarly, when values are expressed as approximations, by use of the antecedent “about,” it will be understood that the particular value is typically more preferred. It will further be understood that the endpoints of each of the ranges are significant both in relation to the other endpoint, and independently of the other endpoint.

“Surface” skin or tissue means the exposed area of skin or tissue associated with a skin ailment, and thus can be below the level of surrounding skin or tissue for deep wounds, ulcers, and other ailments.

“Ailments of the skin” refers to skin disorders, and include, or example, skin lesions, wounds, ulcers, bums, radiation bums, diaper rash, blisters, acne, psoriasis, athlete's foot, and warts.

The term “debridement” as used herein refers to a procedure used the treatment of burns, wounds, and ulcers, wherein the burn, wound, or ulcer is subjected to vigorous mechanical or pharmaceutical cleaning and/or sterilization, which can include treatment with strong anti-microbial agents, or cutting away scabs and dead or damaged tissue.

By the term “effective amount” of a compound or property as provide herein is meant such amount as is capable of performing the function of the compound or property for which an effective amount is expressed. As will be pointed out below, the exact amount required will vary from process to process, depending on recognized variables such as the compounds employed and the processing conditions observed. Thus, it is not possible to specify an exact “effective amount.” However, an appropriate effective amount may be determined by one of ordinary skill in the art using only routine experimentation.

As used herein, the term “topically” means application to the surface of the skin, mucosa, viscera, etc.

As used herein, the term “wound” denotes a bodily injury with disruption of the normal integrity of tissue structures. In one sense the term is intended to encompass the term “surgical site”. In another sense, the term is intended to encompass the terms “sore”, “lesion”, “necrosis” and “ulcer” which may be used interchangeably. Normally, the term “sore” is a popular term for almost any lesion of the skin or mucous membranes and the term “ulcer” is a local defect, or excavation, of the surface of an organ or tissue, which is produced by the sloughing of necrotic tissue. Lesion generally relates to any tissue defect. Necrosis is related to dead tissue resulting from infection, injury, inflammation or infarctions.

As used herein, the term “infection” refers to any microbial invasion of living tissue that is deleterious to the organism. Microbial infections may be caused by microorganisms including, but not limited to, bacteria, viruses and fungi.

As used herein, the term “solvent” refers to any material that is capable of mixing with another solution to extract specific compounds. For example, an amine and a solvent may be added to an aqueous solution, followed by the separation of an immiscible bilayer.

The term “aloe” as used herein generally refers to the viscous gel of the internal portion of the leaf of plants of the genus aloe, and preferably of the species Aloe vera (e.g., Aloe vera Linne), Aloe barbadensis Miller, known in commerce as Curacao Aloe, or of Aloe femx Miller and hybrids of this species with Aloe africana Miller and Aloe spicata Baker, known in commerce as Cape Aloe (Fam. Liliaceae). The gel may be in the form of a liquid concentrate from which water has been removed from gel which has been freshly extracted from the leaf of Aloe vera and to which preservatives have been added to preserve the therapeutic qualities of the gel.

### DISCUSSION

The invention is a solvent composition that is especially effective for treating skin related ailments. The composition, when applied to the skin, promotes skin healing and tissue reconstruction as well as pain relief.

The present invention concerns a composition for the treatment of burns, sunburns, and abrasions. More specifically, the invention relates to a preparation for topical administration having analgesic, antiseptic and skin healing promoting activity. The preparation is particularly suitable for the treatment of burns, scalds and sunburns, and is useful in general in the treatment of any alteration of the dermal tissue.

As it is known, the exposure to an excessive heat of any kind causes on the human epidermis, and often also on the underlying tissues, situations of pathological alteration and lesions the seriousness of which varies according to the duration and the intensity of the exposure, and to the sensitivity of the single individual. Real burns or scalds may be caused, for instance, by the contact with hot matter or articles, such as flames, hot liquids or burning bodies, or also by an excessive exposure to radiation sources, including the sun. According to the current clinical classification, a burn may be of first, second or third degree, depending upon the gravity of the lesion. The first-degree burns are limited to the superficial layers of the epidermis and are characterized by local erythema (redness) and light edema (swelling); the second-degree burns involve a damage extended to the dermis, more marked edema and formation of blisters containing serious exudates, and the third-degree burns are accompanied by a true destruction of the structural elements of the skin, with formation of blisters, sores and the presence, in the most serious cases, of charred zones. In the most critical cases the involvement is extended to general phenomena, such as shock, acute intoxication and anemia.

In all of the cases presented above, in agreement with the various levels of severity, recourse is made to the topical administration of remedies that should exert a range of different actions, including an analgesic action, a stimulating action on the reparative processes of the skin tissues, i.e. an action promoting healing of the lesions, an anti- inflammatory action and, moreover, an antiseptic action, in order to prevent the occurrence of secondary infections on the affected zones. Actually, the injured tissues are particularly prone to the development of infections, which obviously hinder a rapid and complete healing of the skin.

In accordance with the present invention, there is provided herein formulations useful for topical application comprising the extract from cold-processed aloe vera gel, solidified sulfur combined with water based solvent that is discussed further in the following.

In another aspect of this invention provides a composition for treating skin ailments comprising of aloe vera gel, solidified sulfur, and a water based solvent. Wherein the water based solvent is comprised of naturally occurring minerals. The solvent preferably comprises of about 260.4 parts per million of copper along with 400.4 parts per million of calcium. Further comprising of 310 parts per million of sulfur. Additionally, comprising in lesser amounts of gold at 16.5 parts per million, silver at 13.6 parts per million, and iron at 12.2 parts per million.

The composition further contains aloe vera. The aloe vera acts to speed up the healing process on burns to the skin. It provides vital micronutrients to the skin allowing the body to heal the area faster. Also aloe vera has an anti-inflammatory effect on the area around the burn, reducing inflammation and allowing the body to heal the area more quickly without having to work to reduce the inflammation itself. Additionally, the aloe vera has an antimicrobial effect on the damaged area. The antimicrobial effect kills microorganisms around the infected area to reduce the possibility of infection, inflammation, and increase the healing process. Finally, the aloe vera stimulate the skin fibroblasts that allow for scabs to form and healing to begin more quickly with less permanent damager to the skin, such as scarring.

In a further aspect of the composition, a sulfur solid is added. The sulfur is used to treat many different kinds of skin disorders. When applied topically, sulfur acts as an antibacterial agent to help clean the damaged area.

The composition, applied topically, is effective in treating several different types of burns to the skin. The composition forms a gel or paste like texture that can be spread across the damaged area. This will promote the skin through a faster healing process and will minimize scarring around the area. Additionally, the composition works to reduce the pain of the damaged area caused by the burns to the skin.

It will therefore be readily understood by those persons skilled in the art that the present invention is susceptible of a broad utility and application. Many embodiments and adaptations of the present invention other than those herein described, as well as many variations, modifications and equivalent arrangements, will be apparent from or reasonably suggested by the present invention and the foregoing description thereof, without departing from the substance or scope of the present invention. Accordingly, while the present invention has been described herein in detail in relation to its preferred embodiment, it is to be understood that this disclosure is only illustrative and exemplary of the present invention and is made merely for purposes of providing a full and enabling disclosure of the invention. The foregoing disclosure is not intended or to be construed to limit the present invention or otherwise to exclude any such other embodiments, adaptations, variations, modifications and equivalent arrangements, the present invention being limited only by the claims appended hereto and the equivalents thereof.

1. A composition, comprising of a solvent, in association with sulfur, and aloe vera, combined to treat burns incurred to the skin by chemicals, the sun or another occurrence that could cause burns.

2. The composition of claim 1, wherein the combination of the components are to be applied topically.

3. The composition of claim 1, wherein the solvent is water based.

4. The composition on claim 3, wherein a water based solvent comprises of:
a. 400.4 parts per million of Calcium
b. 310 parts per million of Sulfur
c. 260.4 parts per million of Copper
d. 16.5 parts per million of Gold
e. 13.6 parts per million of Silver
f. 12.2 parts per million of Iron

5. The composition of claim 1, wherein the sulfur is in the solidified state.

6. The composition of claim 1, wherein the aloe vera is an extract from the aloe vera plant in the gel or liquid form so as to be mixed with the water based solvent.

7. A method of healing and restoring burned areas of skin, using a composition comprising of a solvent, aloe vera, and sulfur.

8. The method of claim 7, wherein the composition is applied topically to the skin.
